(19) United States
(12) Patent Application Publication
(10) Pub. No.: US 2004/0204721 A1
(43) Pub. Date: Oct. 14, 2004

(54) Hair removal apparatus

(76) Inventor: Kwok Kay Lee, Hong Kong (HK)

Correspondence Address: JACKSON WALKER L.L.P., Suite 2100, 112 E. Pecan Street, San Antonio, TX, 78205, US

(21) Appl. No.: 10/412,383

(22) Filed: Apr. 11, 2003

(51) Int. Cl. A61B 17/50

(52) U.S. Cl. 606/133

(57) ABSTRACT

An apparatus for abrading hair from skin of a body includes an abrasive surface comprising two or more adjacent belts having coplanar flat portions adapted for moving in opposite directions. The body of the apparatus has first and second parallel shafts extending therefrom. The shafts rotate in opposite directions. Belts having an abrasive surface and positioned between the first shaft and second shaft for movement in either the first direction or the second direction.

### BACKGROUND OF THE INVENTION

1. Field of the Invention

The current invention relates to hair removal apparatus and in particular to apparatus for abrading hair from skin.

2. Background Information

The most common methods for removing unwanted hair from the body are by cutting (e.g. shaving) or pulling (e.g. waxing). A less common method of removing unwanted hair is by abrading with an abrasive surface.

U.S. Pat. No. 1,910,647 to Steeg et al, U.S. Pat. No. 2,314,220 to Johnson and U.S. Pat. No. 2,328,886 to Andrews et al all disclose abrasive sheets that fit the hand for manual rubbing of hairy areas of the body to abrade the hair from the skin. These apparatus are time consuming and tiresome for the user.

Automatic abrasive hair removers have been produced which work faster with less effort to the user. U.S. Pat. No. 2,714,788 to Giovanna, U.S. Pat. No. 5,084,046 to Isack and U.S. Pat. No. 5,377,699 to Varnum disclose such apparatus.

U.S. Pat. No. 2,714,788 discloses an automatic apparatus with a rotating abrasive disk that reverses direction automatically. The apparatus has a control for changing rotation speed. However, such an apparatus would be inefficient when operated at low speed and uncontrollable, due to the inertia of the disk changing direction, when operated at high speed.

U.S. Pat. No. 5,084,046 discloses an automatic apparatus with an eccentric abrasive ring. This apparatus can be operated at high speed for better effect, however due to the small size of the abrasive area on the annulus the apparatus is only appears suitable for use on small areas of fine hair.

U.S. Pat. No. 5,377,699 discloses a further automatic apparatus with a rotating abrasive disk or drum. In this apparatus the disk or drum is covered by a foil and does not come into direct contact with the skin surface. This reduces the efficiency of the apparatus and extends the operating time.

### SUMMARY OF THE INVENTION

It is an object of the present invention to provide a hair removal apparatus and in particular an apparatus for abrading hair from the body which is fast and easy to use, and which ameliorates problems with the prior art or at least provides the public with a useful alternative.

According to a first aspect of the invention there is provided an apparatus for abrading hair from skin including an abrasive surface comprising two or more adjacent belts having coplanar flat portions adapted for moving in opposite directions.

According to a second aspect of the invention there is provided a hair removal apparatus including:

a body,

a first shaft extending from the body,

a second shaft extending from the body and parallel to the first shaft,

means for rotating the first shaft in a first direction and rotating the second shaft in a second direction,

two or more belts having an abrasive surface and positioned between the first shaft and second shaft for movement in either the first direction or the second direction, and in which each belt moves in the direction different to the belt or belts adjacent to it.

Preferably, the hair removal apparatus includes two or more pulleys positioned on each of the first and second shafts for receiving the belts.

Preferably, the belts have adjacent flat spans between the first shaft and second shaft providing an abrasive surface therebetween.

According to a third aspect of the invention there is provided a hair removal apparatus including:

a body,

a first shaft extending from the body and having a first gear positioned on it,

a second shaft extending from the body parallel to the first shaft and having a second gear positioned on it,

a motor positioned in the body and rotating an output shaft,

a third gear position on the output shaft and engaging the first gear for rotating the first shaft in a first direction,

a fourth gear engaging the third gear and the second gear for rotating the second shaft in a second direction,

two or more belts having an abrasive surface and positioned between the first shaft and second shaft for movement in either the first direction or the second direction, and in which each belt moves in the direction different to the belt or belts adjacent to it.

According to a forth aspect of the invention there is provided a hair removal apparatus including:

a body,

a first shaft extending from the body,

a second shaft extending from the body and parallel to the first shaft,

means for rotating the first shaft in a first direction and rotating the second shaft in a second direction,

two or more pulleys positioned on each of the first and second shafts,

two or more belts having an abrasive surface and positioned between pulleys on the first shaft and second shaft for forming a flat span therebetween and for movement in either the first direction or the second direction, and in which each belt moves in the direction different to the belt or belts adjacent to it.

Further aspects of the invention will become apparent from the following description, which is given by way of example only.

### BRIEF DESCRIPTION OF THE DRAWINGS

Embodiments of the invention will now be described with reference to the accompanying drawings in which:

FIG. 1 is a first perspective view of a hair removal apparatus according to the invention,

FIG. 2 is a second perspective view of the hair removal apparatus with a body portion removed,

FIG. 3 is a plan view of FIG. 2,

FIG. 4 is an elevation view of FIG. 2,

FIG. 5 illustrates of a gear arrangement for the hair removal apparatus, and

FIGS. 6 and 7 are section views through adjacent belts of the apparatus.

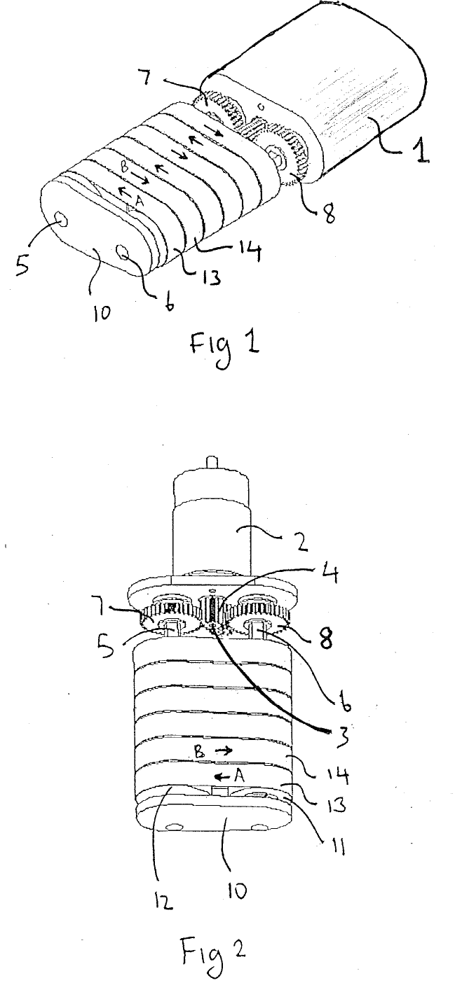

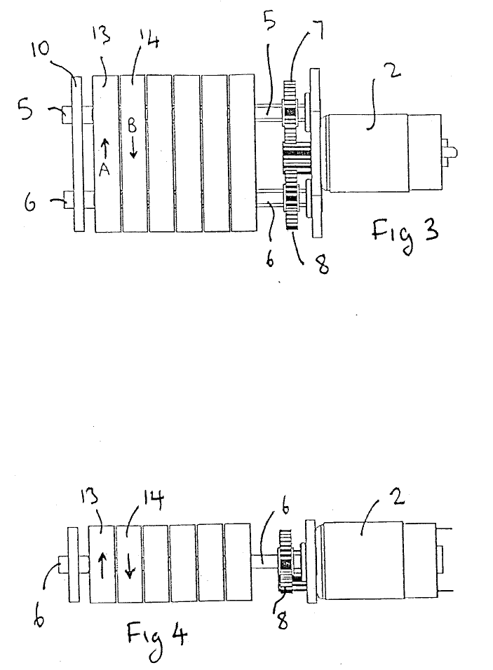

### DESCRIPTION OF THE PREFERRED EMBODIMENTS

A preferred embodiment of a hair removal apparatus according to the invention includes a body 1 enclosing a motor 2 with a drive shaft 3. A drive gear 4 is positioned on the drive shaft 3. First and second shafts 5, 6 extend from the body 1 parallel to each other. Providing support to the distal ends of the shafts 5, 6 is a link 10.

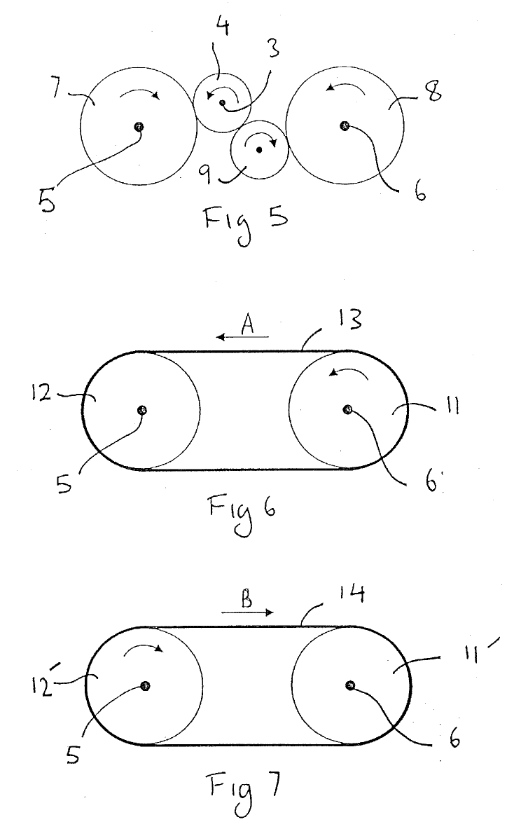

Positioned on each shaft 5, 6 proximate the body 1 are second and third gears 7 and 8. Drive gear 4 meshes with second gear 7 to drive first shaft 5 in a first direction. A second drive gear 9 is located between drive gear 4 and third gear 8 of second shaft 6. Second drive gear 9 meshes with drive gear 4 and third gear 8 to cause second shaft 6 to move in the opposite direction to first shaft 5. The arrangement of the four gears 4, 7, 8 and 9 is illustrated in FIG. 5.

Referring to FIG. 5, drive gear 4 is positioned on motor shaft 3 and rotates in an anti-clockwise direction. It meshes with first shaft gear 7 and causes first shaft 5 to move in a clockwise direction A. Second drive gear 9 is positioned to mesh with drive gear 4 and gear 8 of second shaft 6. Anti-clockwise rotation of drive gear 4 causes second drive gear 9 to rotate in a clockwise direction and second shaft 6 to move in an anti-clockwise direction B.

In an alternative embodiment a different gear arrangement is utilised to move the two shafts 5, 6 in opposite directions. In this arrangement motor 3 drives first shaft 5 directly. First shaft gear 7 meshes with second shaft gear 8 turning it in the opposite direction. There are no drive gears 4, 9. Other arrangements are also envisaged and such are well within the capability of the skilled addressee.

Positioned on each of first and second shafts 5, 6 are a plurality of coaxial pulleys 11, 12. In the preferred embodiment there are 6 coaxial pulleys on each shaft 5, 6. However, there may be as few as two and as many as can be practically accommodated. Positioned between corresponding pulleys 11, 12 on opposite shafts 5, 6 are a plurality of belts 13, 14 etc. The belts have an abrasive external surface and extend in a flat span between the pulleys 11, 12. The plurality of adjacent belts, six in the preferred embodiment, provides a large working surface for abrading hair from the skin.

Alternate pulleys 12 along shaft 5, and alternate pulleys 11 along shaft 6, are fixed to the shaft to move adjacent belts 13, 14 in opposite directions A, B at the abrading surface.

FIGS. 6 and 7 illustrate a section through belts 13 and 14 respectively. In FIG. 6 the pulley 11 on second shaft 6 is fixed to the shaft in order to move belt 13 in the anti-clockwise direction A. Corresponding pulley 12 on first shaft 5 is free to rotate with respect to shaft 5. Referring to FIG. 7, in the immediately adjacent belt 14 the pulley 11′ is free to rotate about second shaft 6 and the corresponding pulley 12′ on first shaft 5 is fixed for rotation with the shaft thus moving the adjacent belt 14 in the clockwise direction B.

The invention provides an apparatus with an abrasive surface comprising two or more adjacent belts 13, 14 having co-planner flat portions adapted to move in opposite directions A, B. The large abrasive surface works a large area of hair on the skin at one time. The two or more adjacent belts 13, 14 moving an opposite directions A, B provide additional working of the hair in different directions with little effort from the user. At the junction between adjacent belts 13, 14 moving in opposite directions A, B the hair is caused to twist and mat and the abrading affect is improved thus reducing the time taken to remove the hair.

Where in the foregoing description reference has been made to integers or elements having known equivalents then such are included as if individually set forth herein.

Embodiments of the invention have been described, however it is understood that variations, improvements or modifications can take place without departure from the spirit of the invention or scope of the appended claims.

What is claimed is:

1. An apparatus for abrading hair from skin including an abrasive surface comprising two or more adjacent belts having coplanar flat portions adapted for moving in opposite directions.

2. A hair removal apparatus including:
a body,
a first shaft extending from the body,
a second shaft extending from the body and parallel to the first shaft,
means for rotating the first shaft in a first direction and rotating the second shaft in a second direction,
two or more belts having an abrasive surface and positioned between the first shaft and second shaft for movement in either the first direction or the second direction, and in which each belt moves in the direction different to the belt or belts adjacent to it.

3. The apparatus of claim 2 including two or more pulleys positioned on each of the first and second shafts for receiving the belts.

4. The apparatus of claim 2 wherein the belts have adjacent flat spans between the first shaft and second shaft providing an abrasive surface therebetween.

5. A hair removal apparatus including:
a body,
a first shaft extending from the body and having first a gear positioned on it,
a second shaft extending from the body parallel to the first shaft and having a second gear positioned on it,
a motor positioned in the body and rotating an output shaft,
a third gear position on the output shaft and engaging the first gear for rotating the first shaft in a first direction,
a fourth gear engaging the third gear and the second gear for rotating the second shaft in a second direction,
two or more belts having an abrasive surface and positioned between the first shaft and second shaft for movement in either the first direction or the second direction, and in which each belt moves in the direction different to the belt or belts adjacent to it.

6. A hair removal apparatus including:
a body,
a first shaft extending from the body,
a second shaft extending from the body and parallel to the first shaft,
means for rotating the first shaft in a first direction and rotating the second shaft in a second direction,
two or more pulleys positioned on each of the first and second shafts,
two or more belts having an abrasive surface and positioned between pulleys on the first shaft and second shaft for forming a flat span therebetween and for movement in either the first direction or the second direction, and in which each belt moves in the direction different to the belt or belts adjacent to it.
